(19) United States
(12) Patent Application Publication
(10) Pub. No.: US 2018/0184881 A1
(43) Pub. Date: Jul. 5, 2018

(54) ENDOSCOPE PROCESSOR

(71) Applicant: OLYMPUS CORPORATION, Tokyo (JP)

(72) Inventors: Takeshi URASAKI, Tokyo (JP); Aki MATSUMOTO, Sagamihara-shi (JP); Nobuyasu ITO, Tokyo (JP); Shinichiro SAEKI, Tokyo (JP); Kenichiro MATSUSHITA, Sagamihara-shi (JP); Hideyuki WADA, Tokyo (JP)

(73) Assignee: OLYMPUS CORPORATION, Tokyo (JP)

(21) Appl. No.: 15/903,209

(22) Filed: Feb. 23, 2018

Related U.S. Application Data
(63) Continuation of application No. PCT/JP2016/074220, filed on Aug. 19, 2016.

(30) Foreign Application Priority Data
Aug. 27, 2015 (JP) 2015-168027

(51) Int. Cl. A61B 1/00; A61B 1/045

(52) U.S. Cl. CPC A61B 1/00006; A61B 1/0002; A61B 1/00048; A61B 1/045; A61B 1/00039

(57) ABSTRACT

An endoscope system includes a memory configured to be able to store a parameter set which is a combination of parameters associated with various processes during an endoscope inspection and set for each user, the memory being able to store a number of parameter sets corresponding to a plurality of user accounts, a touch panel configured to selectively display a display screen made up of a plurality of layers and a control section configured to cause the touch panel to display a user setting selection list for calling the parameter set for each user stored in the memory for part of a display region of a main screen that classifies and displays functions with high use frequency during the endoscope inspection.

### CROSS REFERENCE TO RELATED APPLICATION

This application is a continuation application of PCT/JP2016/074220 filed on Aug. 19, 2016 and claims benefit of Japanese Application No. 2015-168027 filed in Japan on Aug. 27, 2015, the entire contents of each of which are incorporated herein by their reference.

### BACKGROUND OF THE INVENTION

### 1. Field of the Invention

The present invention relates to an endoscope processor, and more particularly, to an endoscope processor capable of registering a plurality of pieces of user setting information.

### 2. Description of the Related Art

Conventionally, endoscopes provided with an image pickup device have been widely used in medical and industrial fields. Techniques for carrying out various signal processes associated with the endoscopes using a signal processing apparatus detachably connected to the endoscope and called a “video processor” and configuring an endoscope system are also well known.

Among video processors carrying out various signal processes as described above, video processors having a function of setting and registering various parameters associated with image processing for each user are also well known. More specifically, a video processor is known which is provided with a function of setting and registering, for each user, various parameters such as adjustment of an endoscope image (image brightness, color tone, chroma, contrast or the like) for each observation mode (normal light observation mode, NBI observation mode or the like) in addition to an electronic shutter speed, a photometric area or light-adjusting sensitivity of the endoscope connected.

Here, in the video processor which has the function of setting/registering various parameters for each user as described above, a certain operation is required to call an arbitrary user setting among registered user settings.

More specifically, for example, with a video processor described in the specification of Japanese Patent No. 4575042, through the following operations, the setting associated with the registered user is called:

(1) First, a “setting key” of a keyboard is pressed;

(2) A “list of various settings” is displayed on a display screen and a “setting button” to call a registered user is selected and pressed;

(3) Since a “user setting list” is displayed, a “user button” to be called is selected; and

(4) A “call button” to call the selected user is pressed.

The operation of selecting the registered user setting is preferably done quickly before starting to use the endoscope system (before an inspection starts).

### SUMMARY OF THE INVENTION

An endoscope processor according to one aspect of the present invention includes a storage section configured to be able to store a parameter set which is a combination of parameters associated with various processes during an endoscope inspection and set for each user, the storage section being able to store a number of parameter sets corresponding to a plurality of user accounts, a display section configured to selectively display a display screen made up of a plurality of layers and a display control section configured to cause the display section to display a user setting selection list for calling the parameter set for each user stored in the storage section for part of a display region of a main screen that classifies and displays functions with high use frequency during the endoscope inspection.

### BRIEF DESCRIPTION OF THE DRAWINGS

FIG. 1 is a perspective view illustrating a schematic configuration of an endoscope system according to a first embodiment of the present invention;

FIG. 2 is a block diagram illustrating a functional configuration of main parts in the endoscope system according to the first embodiment;

FIG. 3 is a diagram illustrating a display example of a touch panel of a video processor in the endoscope system according to the first embodiment;

FIG. 4 is a diagram illustrating a status display section region on the touch panel in the endoscope system according to the first embodiment;

FIG. 5 is a diagram illustrating a functional classification switching display section region on the touch panel in the endoscope system according to the first embodiment;

FIG. 6 is a diagram illustrating the function operation portion region on the touch panel in the endoscope system according to the first embodiment;

FIG. 7 is a diagram illustrating an example of a user setting selection list on the touch panel in the endoscope system according to the first embodiment;

FIG. 8 is a diagram illustrating a screen example in a process of calling a user setting on the touch panel in the endoscope system according to the first embodiment;

FIG. 9 is a diagram illustrating a screen example when a user setting is called on the touch panel in the endoscope system according to the first embodiment;

FIG. 10 is a diagram illustrating a display example of a touch panel of a video processor in an endoscope system according to a second embodiment of the present invention;

FIG. 11 is a diagram illustrating a display example of a touch panel of a video processor in an endoscope system according to a third embodiment of the present invention;

FIG. 12A is diagram illustrating a display example of the touch panel of the video processor in the endoscope system according to the third embodiment;

FIG. 12B is a diagram illustrating a display example of the touch panel when a home button is pressed on the touch panel in the endoscope system according to the third embodiment;

FIG. 12C is a diagram illustrating a display example of the touch panel when an image browsing button is pressed on the touch panel in the endoscope system according to the third embodiment;

FIG. 12D is a diagram illustrating a display example of the touch panel when a setting button is pressed on the touch panel in the endoscope system according to the third embodiment;

FIG. 13 is a diagram illustrating a display example of a touch panel of a video processor in an endoscope system according to a fourth embodiment of the present invention;

FIG. 14 is a diagram illustrating another display example of the touch panel of the video processor in the endoscope system according to the fourth embodiment;

FIG. 15 is a diagram illustrating a display example of a touch panel of a video processor in an endoscope system according to a fifth embodiment of the present invention;

FIG. 16A is a diagram illustrating a video processor in an endoscope system according to a sixth embodiment of the present invention;

FIG. 16B is a diagram illustrating an observation monitor in an endoscope system according to a sixth embodiment of the present invention; and

FIG. 17 is a diagram illustrating a situation in which the display of the observation monitor is changed through a predetermined operation of the touch panel of the video processor in the endoscope system according to the sixth embodiment.

### DETAILED DESCRIPTION OF THE PREFERRED EMBODIMENTS

Hereinafter, embodiments of the present invention will be described with reference to the accompanying drawings.

The present invention is not limited by the embodiments. In the description of drawings, the same parts are assigned the same reference numerals. It should be noted that the drawings are schematic representations and may be different from the reality. The drawings include components which differ in size and ratio among the drawings.

### First Embodiment

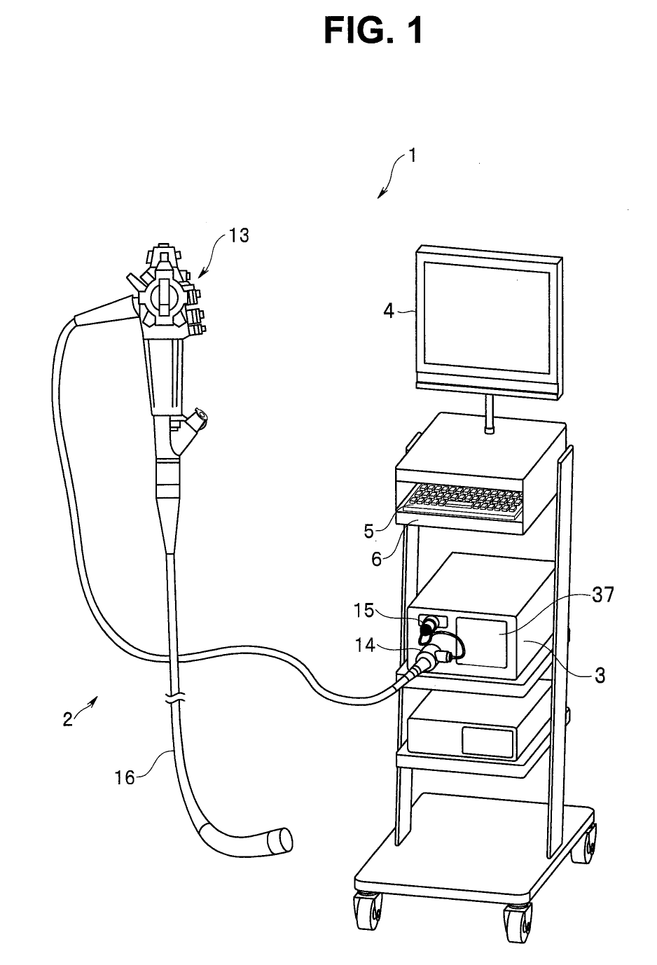

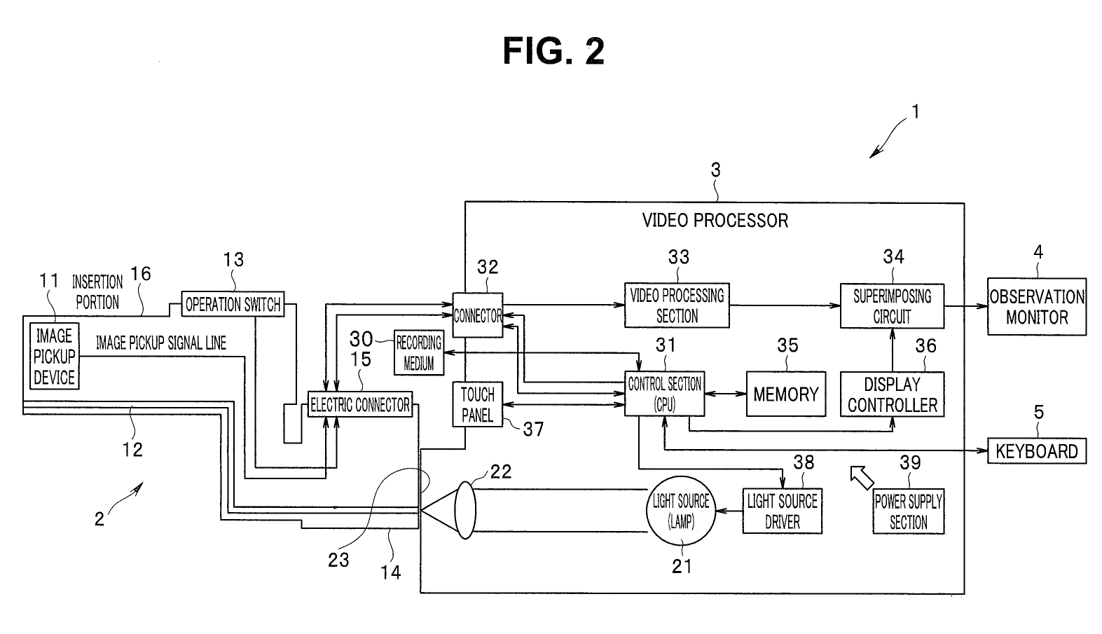

FIG. 1 is a perspective view illustrating a schematic configuration of an endoscope system according to a first embodiment of the present invention and FIG. 2 is a block diagram illustrating a functional configuration of main parts in the endoscope system according to the first embodiment.

As shown in FIG. 1, an endoscope system 1 is mainly constructed of an endoscope 2 configured to insert the distal end portion into a lumen in a body cavity of a subject, thereby pick up an in-vivo image of an object and output an image signal of the object image, a video processor 3 configured to apply predetermined image processing to the image signal outputted from the endoscope 2, include a light source function of generating illumination light to be emitted from the distal end of the endoscope 2 and further comprehensively control operation of the entire endoscope system 1, an observation monitor 4 configured to display the image subjected to the image processing in the video processor 3 and a keyboard 5 connected to the video processor 3 for inputting an operation instruction or character information or the like.

Note that in the endoscope system 1 of the present embodiment, although the video processor 3 is provided with the aforementioned light source function, without being limited to the configuration, a light source apparatus having the light source function may be configured as a component independent of the video processor 3. Although a configuration adopting a simultaneous scheme for the endoscope system 1 will be described in the present embodiment, a frame-sequential scheme is also applicable in the present invention.

The endoscope 2 includes a flexible and elongated insertion portion 16, an image pickup device 11 provided at a distal end portion of the insertion portion 16 and configured to pick up an image of a subject, a light guide 12 configured to guide illumination light to the distal end of the insertion portion, an operation switch 13 provided at an operation portion configured to operate the endoscope 2, a connector portion 14 for connection with a light source connector 23 in the video processor 3 and an electric connector 15 extending from the connector portion 14 for connection with a connector 32 in the video processor 3.

Note that the endoscope 2 may be a so-called rigid endoscope used for surgery. A CMOS (complementary metal oxide semiconductor) image sensor is adopted for the image pickup device 11 in the present embodiment, but a CCD image sensor may also be adopted. Moreover, although the image pickup device 11 is provided at the distal end portion of the insertion portion in the present embodiment, without being limited to the configuration, for example, a configuration may be adopted in which the image pickup device 11 is provided inside the operation portion where the operation switch 13 is provided and an optical image is transmitted from the distal end portion of the insertion portion to the image pickup device 11 in the operation portion using an image guide fiber.

Furthermore, although the video processor 3 is configured to be connected to the endoscope 2 in the present embodiment, without being limited to the configuration, a configuration may also be adopted in which the video processor 3 is connected to a camera head mounted on an eyepiece part of an optical endoscope (fiber scope or optical viewing tube for surgery) which is inserted into a body cavity.

Furthermore, in the present embodiment, the endoscope 2 and the video processor 3 are connected via the electric connector 15 and the connector 32 so as to transmit an electric signal by wire, but without being limited to the configuration, a configuration may be adopted in which an electric signal is transmitted wirelessly.

The video processor 3 as a signal processing apparatus is constructed of the connector 32 connected to the electric connector 15 of the endoscope 2, a control section 31 configured to comprehensively control operation of the entire endoscope system 1, a memory 35 connected to the control section 31 to store various types of information, a video processing section 33 configured to apply predetermined signal processing to an image pickup signal from the image pickup device 11 inputted via the connector 32, a display controller 36 configured to change a character size of character information displayed on the observation monitor 4, a superimposing circuit 34 configured to superimpose the character information from the display controller 36 on a video signal outputted from the video processing section 33, a touch panel 37 configured to perform various operations and settings and a power supply section 39 configured to supply power to operate each circuit section at least in the video processor 3.

The control section 31 is a CPU configured to comprehensively control operation of the entire endoscope system 1 and is configured not only to control the image pickup device 11 in the endoscope 2 connected to the video processor 3 but also to have a function as an initial state detection section configured to detect that the video processor 3 is set in an initial state.

Upon detecting that the video processor 3 is set in the initial state, the control section 31 also performs a function as a display control section configured to cause the touch panel 37 which is a display section to display an initial screen.

The power supply section 39 performs a function as a power supply to supply power for operating the electronic device such as each circuit section in the endoscope 2 connected to the video processor 3 in addition to the aforementioned electronic device such as each circuit section in the video processor 3.

The video processing section 33 is configured to apply predetermined image processing, that is, noise reduction processing, white balance processing, color correction or the like to the video signal from the image pickup device 11 and output the obtained video signal to the superimposing circuit 34.

The memory 35 is implemented using a semiconductor memory and is configured to store various programs for operating the endoscope system 1 including the video processor 3 and the endoscope 2 and data including various parameter sets necessary to operate the endoscope system 1.

Note that the “parameter set” is, for example, a combination of various parameters associated with image processing set for each user in the present embodiment. The memory 35 performs a function as a storage area that can store parameter sets corresponding to a plurality of user accounts (details will be described later).

The display controller 36 outputs predetermined character information to be displayed as a so-called OSD (on-screen display) on the observation monitor 4 under the control of the control section 31 to the superimposing circuit 34. At this time, the display controller 36 changes the size of the character information based on the control of the control section 31 and outputs the character information to the superimposing circuit 34.

For example, when the user operates (presses) the operation switch 13 of the endoscope 2, an operation signal is supplied to the control section 31. Upon receiving the operation signal, the control section 31 controls the display controller 36 so as to change the size of the character information.

The superimposing circuit 34 as an image generation section generates an endoscope inspection image which is the character information from the display controller 36 superimposed on the video signal (endoscope image) from the video processing section 33 and outputs the endoscope inspection image to the observation monitor 4. In this way, the predetermined endoscope inspection image is displayed on the observation monitor 4.

In the present embodiment, the video processor 3 is provided with a light source function of generating illumination light to be emitted from the distal end of the endoscope 2. That is, the video processor 3 includes a light source 21 configured to generate illumination light, a light source driver 38 configured to drive the light source 21 under the control of the control section 31 and a condensing lens 22 configured to condense the illumination light from the light source 21 on an incident end face of the light guide 12.

The light source 21 is configured using a white LED (light emitting diode) or xenon lamp and generates white light, driven and controlled by the light source driver 38.

The light source driver 38 supplies a current to the light source 21 to thereby cause the light source 21 to generate white light. The light generated by the light source 21 is radiated from a tip of the distal end portion of the insertion portion of the endoscope 2 via the condensing lens 22 and the light guide 12.

The observation monitor 4 has a function of receiving an in-vivo image generated by the video processor 3 via a video cable from the video processor 3 and displaying the in-vivo image, and is constructed using liquid crystal or organic EL (electro luminescence) or the like.

The keyboard 5 is connected to the video processor 3 and has a function of inputting a predetermined operation instruction or character information or the like.

### Description of Menu Screen on Touch Panel

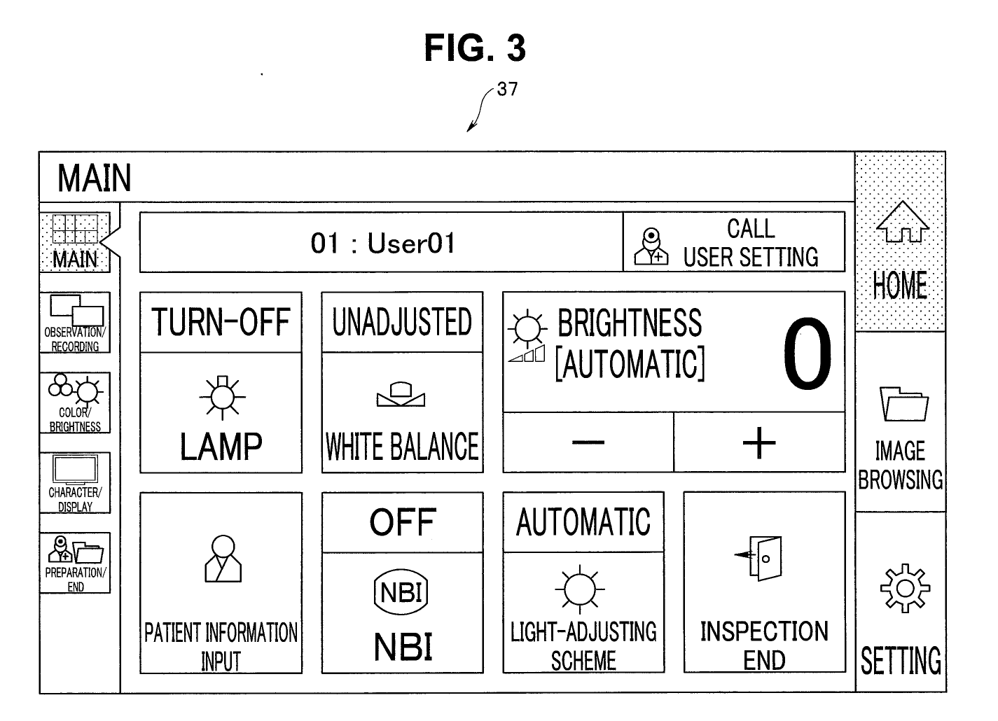

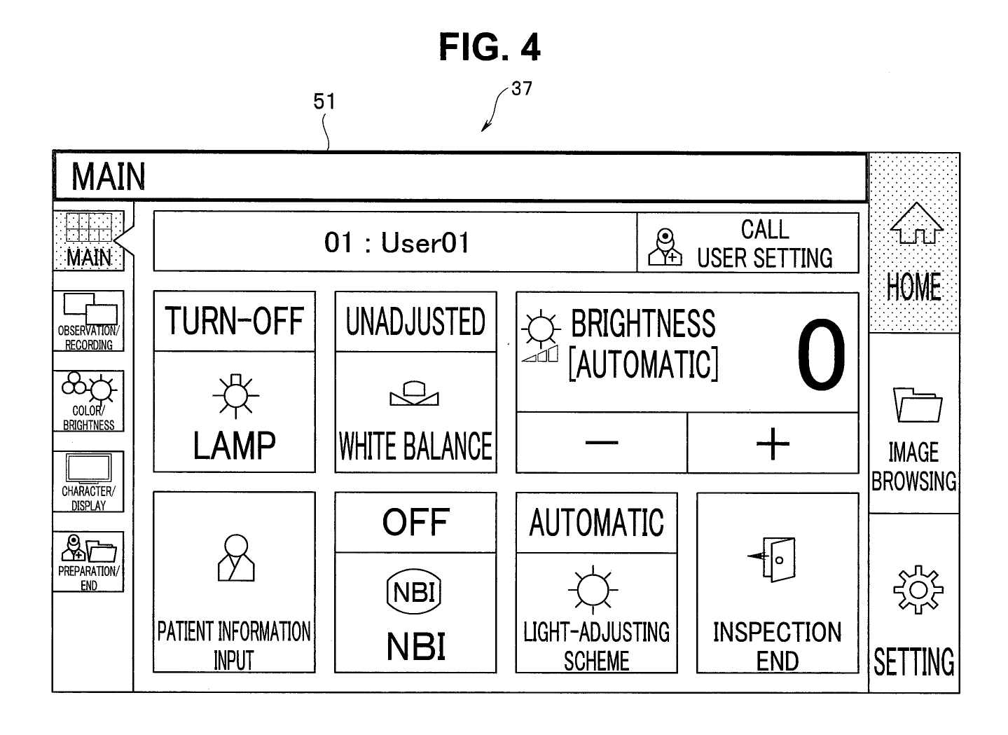

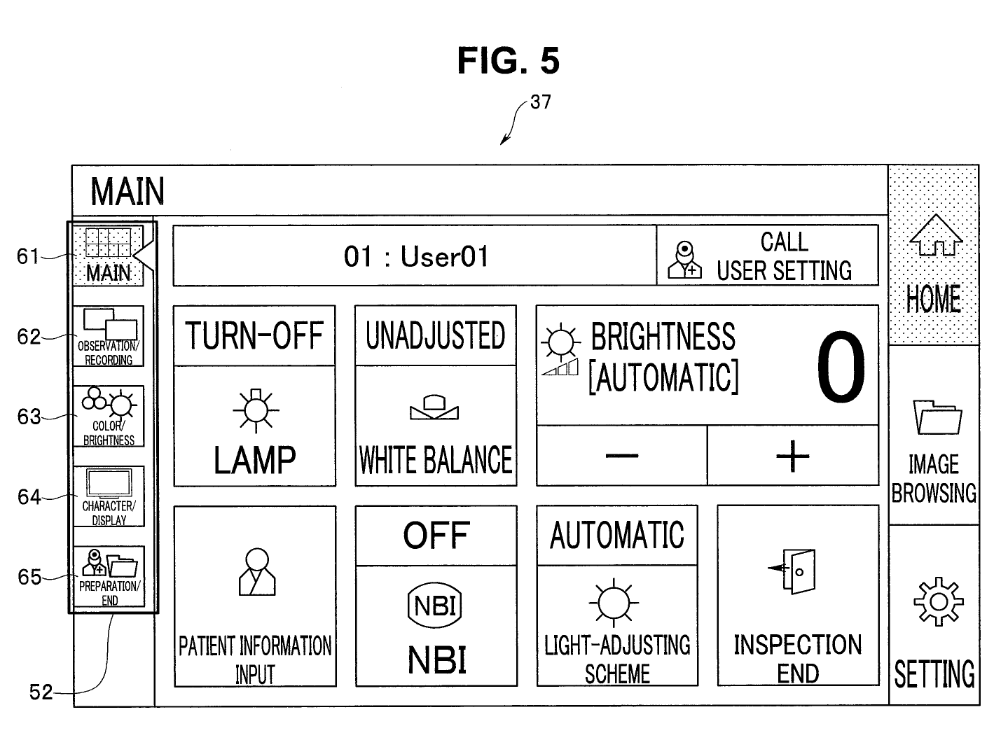

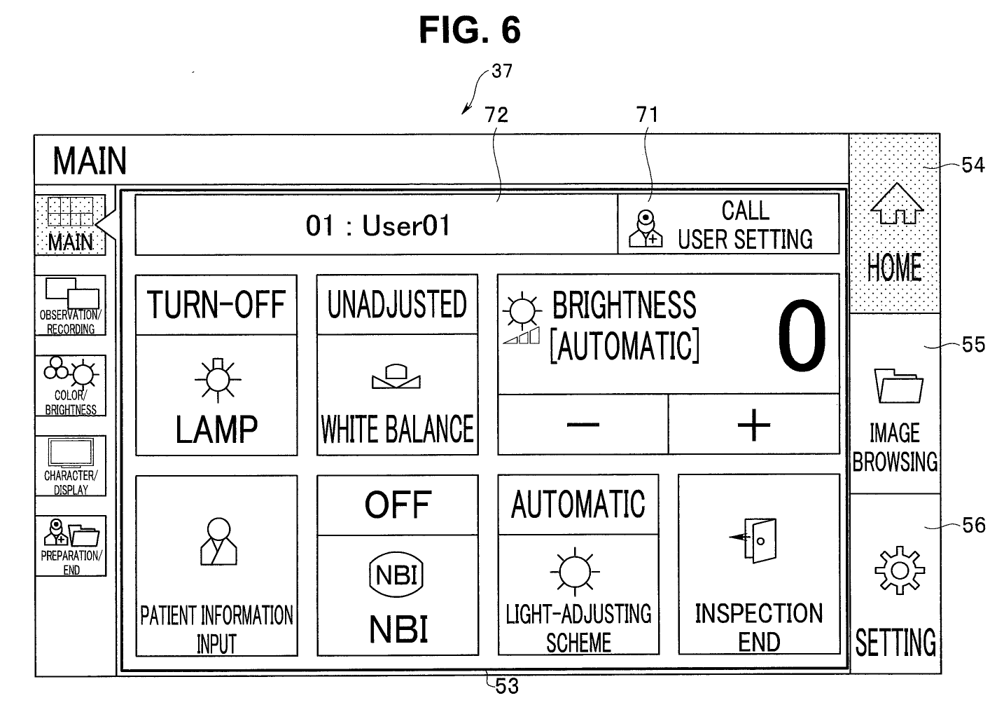

FIG. 3 is a diagram illustrating a display example of the touch panel of the video processor in the endoscope system according to the first embodiment. FIG. 4 is a diagram illustrating a status display section region on the touch panel, FIG. 5 is a diagram illustrating a functional classification switching display section region on the touch panel and FIG. 6 is a diagram illustrating a function operation portion region on the touch panel.

As described above, the video processor 3 disposes the touch panel 37 on the front face of the housing apparatus body. The touch panel 37 is constructed of a display section made up of a liquid crystal panel and a position information input section made up of a touch pad, and configured to perform various operations and settings under the control of the control section 31 which will be described later.

Note that according to the present embodiment, a so-called capacitive type touch panel is adopted for the touch panel 37, but without being limited to the configuration, the touch panel 37 may be configured under a so-called resistive film scheme.

As shown in FIG. 4, FIG. 5 and FIG. 6, a status display section region 51, a function classification switching display section region 52, a function operation portion region 53, a home button 54, an image browsing button 55 and a setting button 56 are disposed on the display screen of the touch panel 37.

As shown in FIG. 4, the status display section region 51 is a region in which the name of a screen currently being displayed or the state of the video processor 3 is displayed using an icon or the like. FIG. 4 shows that the screen currently being displayed is a “main” screen.

As shown in FIG. 5, the function classification switching display section region 52 is a region where a plurality of buttons to switch among various operation functions and display the operation functions are classified and displayed, and in the present embodiment, a main button 61, an observation/recording button 62, a color/brightness button 63, a character/display button 64, a preparation/end button 65 are arranged.

When pressed (touched), the main button 61 displays the “main” screen. The main screen is a screen that classifies and displays functions with high use frequency from among the various functions.

When pressed (touched), the observation/recording button 62 displays an “observation/recording” screen. The observation/recording screen is a screen that classifies and displays functions associated with observation/recording from among the various functions.

When pressed (touched), the color/brightness button 63 displays a “color/brightness” screen. The color/brightness screen is a screen that classifies and displays functions associated with color/brightness from among the various functions.

When pressed (touched), the character/display button 64 displays a “character/display” screen. The character/display screen is a screen that classifies and displays functions associated with character information to be displayed on the observation monitor 4 or information on the camera head connected to the video processor 3 from among the various functions.

When pressed (touched), the preparation/end button 65 displays a “preparation/end” screen. The preparation/end screen is a screen that classifies and displays functions to be used to make preparations or end an inspection from among the various functions.

As shown in FIG. 6, the function operation portion region 53 is a screen that displays buttons (function operation buttons) or the like for operating various functions and the various function operation button groups are switched through a pressing operation (a touching operation) of each button in the function classification switching display section region 52.

Note that when the control section 31 detects that the video processor 3 is set to an initial state (e.g., immediately after power is turned on), an initial screen is displayed on the touch panel 37 under the control of the control section 31. FIG. 6 illustrates an example of the initial screen.

The present embodiment displays the “main” screen that classifies and displays functions with high use frequency on the touch panel 37 as the initial screen as shown in FIG. 6. Furthermore, the present embodiment arranges a user setting information call button 71 to select a parameter set to be used when the video processor 3 is used for part of the function operation portion region 53 on the main screen as the initial screen.

On the other hand, as shown in FIG. 6, the home button 54, the image browsing button 55 and the setting button 56 are disposed on a side of the function operation portion region 53.

When pressed (touched), the home button 54 is configured to display a screen to be used during an endoscope inspection.

When pressed (touched), the image browsing button 55 is configured to display a screen to be used for image browsing including data arrangement after the endoscope inspection.

When pressed (touched), the setting button 56 is configured to display a screen from which the user can perform user setting, maintenance or system setting.

Here, the present embodiment is configured to be able to set and register a parameter set which is a combination of various parameters or the like associated with image processing for a plurality of users. For example, by operating the setting button 56, the present embodiment is configured to be able to set and register a parameter set for a plurality of users.

In this case, data of the parameter set which is set for each user is stored in the memory 35 under the control of the control section 31. Note that examples of such parameters include an electronic shutter speed, a photometric area, light-adjusting sensitivity as well as adjustment of an endoscope image (image brightness, color tone, chroma, contrast or the like) for each observation mode (normal light observation mode, NBI observation mode or the like) in the endoscope 2 connected.

Hereinafter, the description of the present embodiment will be continued on the assumption that a parameter set for each user is registered in advance through the aforementioned setting operation.

Returning to FIG. 6, a user display section 72 showing the currently selected user name is arranged in the vicinity of the user setting information call button 71. Now, the user display section 72 displays “01: User 01” indicating that “user 01” is selected.

Furthermore, at this time, the function operation portion region 53 displays a function operation portion corresponding to the selected “user 01.” That is, a function operation portion associated with the state of the illumination lamp, white balance and brightness adjustment in accordance with the parameter set associated with the preset/registered “user 01.”

Next, a procedure for the user to call a setting associated with another registered user will be described.

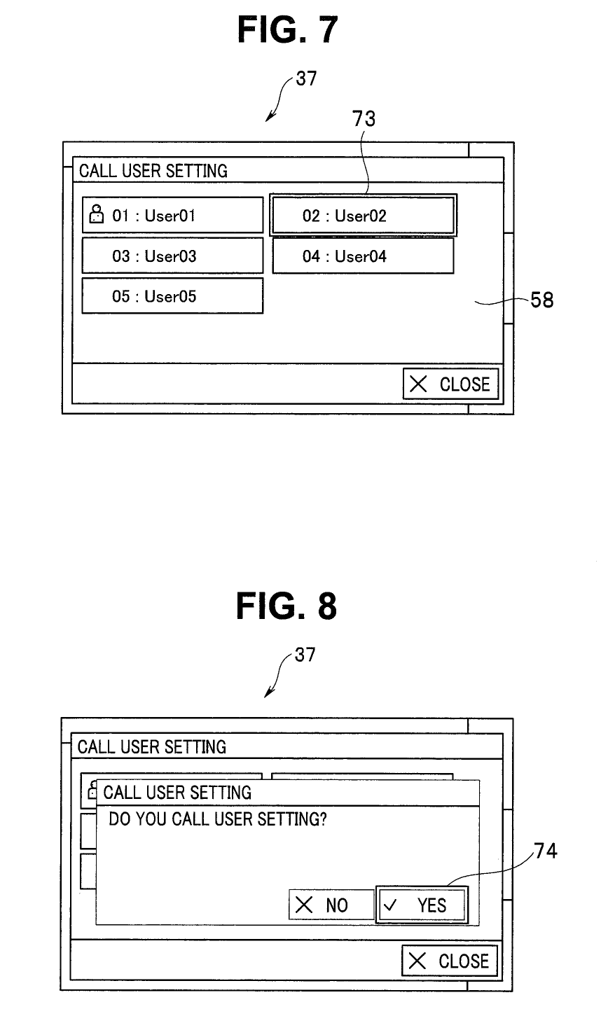

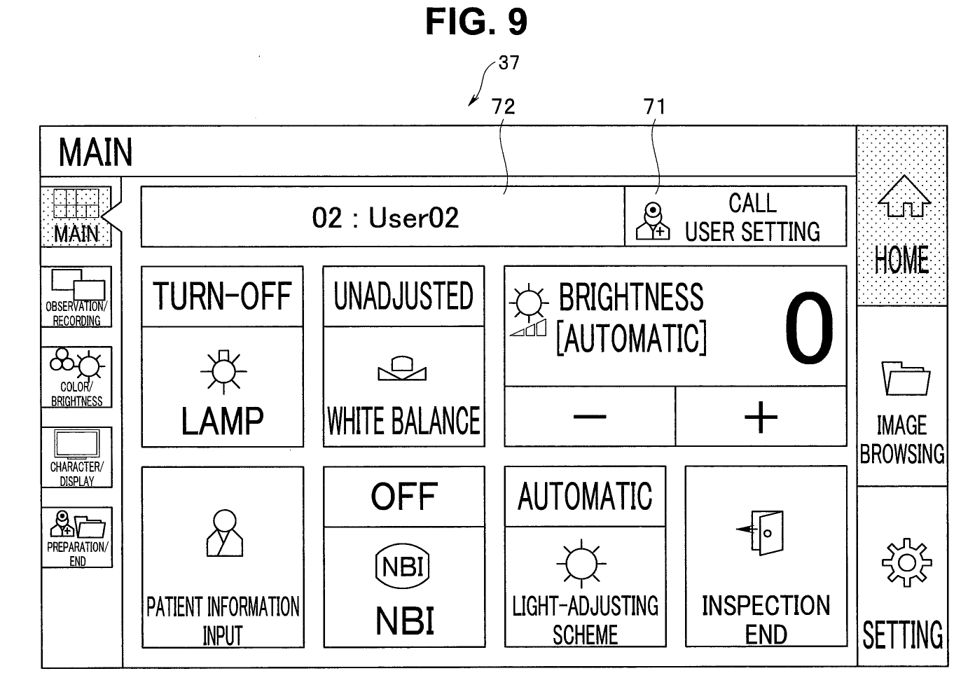

FIG. 7 is a diagram illustrating an example of a user setting selection list on the touch panel in the endoscope system according to the first embodiment. FIG. 8 is a diagram illustrating a screen example in a process of calling a user setting on the touch panel and FIG. 9 is a diagram illustrating a screen example when a user setting is called on the touch panel.

In the present embodiment, the user can change the screen to a screen for calling another user setting by pressing (touching) the user setting information call button 71 displayed on the main screen of the touch panel 37.

That is, when the user setting information call button 71 is operated, the display screen of the touch panel 37 shows a currently registered user setting selection list 58 as shown in FIG. 7.

Here, if a “02: User 02” button 73 for selecting “user 02” is pressed from the user setting selection list 58 as shown in FIG. 7, a confirmation screen shown in FIG. 8 is displayed, and after a confirmation button 74 is pressed, a function operation portion corresponding to the selected “user 02” is displayed in the function operation portion region 53 as shown in FIG. 9.

The user display section 72 displays the currently selected user name, that is “02: User 02” indicating the “user 02” in the current case.

The present embodiment provides an effect, when calling a user setting which is set and registered in advance, that it is possible to easily call the user setting without requiring any complicated operation as in the prior art.

Note that the present embodiment has provided a confirmation screen to prompt the user to do confirmation in the process of calling the user setting as shown in FIG. 8, but a setting may also be made in advance so as to omit the confirmation operation.

When an arbitrary user setting is selected from the user setting selection list 58, omission of the confirmation screen allows a user setting which is set and registered in advance to be called more quickly.

### Second Embodiment

Next, a second embodiment of the present invention will be described.

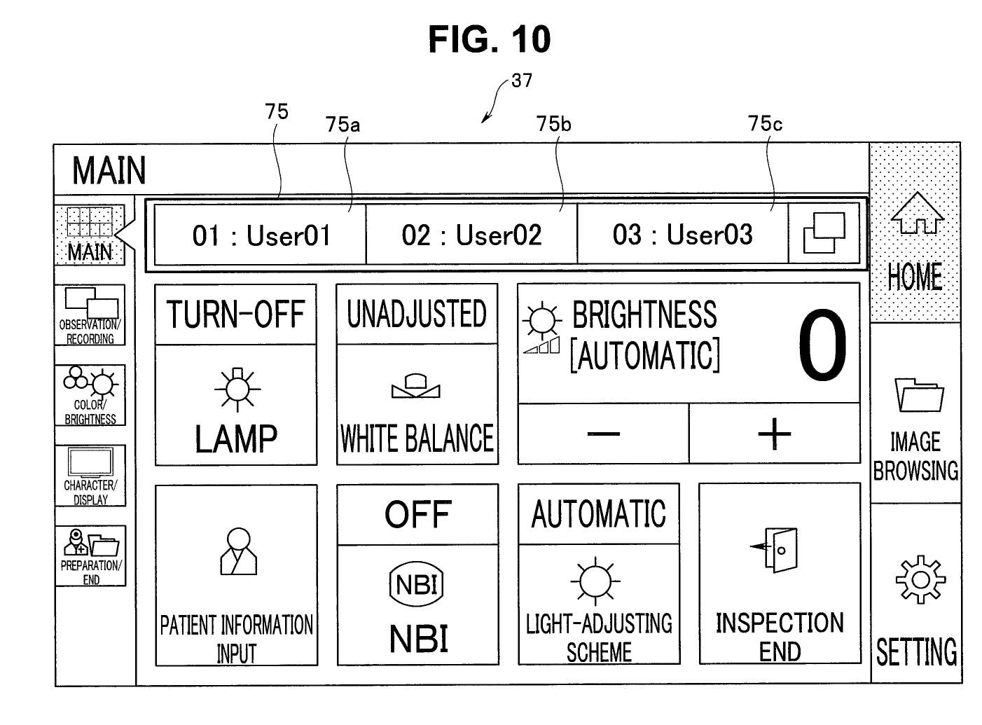

FIG. 10 is a diagram illustrating a display example of the touch panel of a video processor in an endoscope system according to the second embodiment of the present invention.

The endoscope system according to the present second embodiment has basically the same configuration as the configuration of the first embodiment, and so only differences from the first embodiment will be described here and description of other details will be omitted.

According to the first embodiment, the user setting information call button 71 and the user display section 72 are provided in the function operation portion region 53 of the touch panel 37 in the video processor 3, the screen is changed to a screen displaying the user setting selection list 58 by pressing the user setting information call button 71 (see FIG. 7), and an arbitrary user setting is selected from the user setting selection list 58 so as to call the user setting which is set and registered in advance.

In contrast, in the video processor 3 according to the present second embodiment, the function operation portion region 53 of the touch panel 37 is provided with a user setting call button 75 for directly calling the user setting instead of the aforementioned user setting information call button 71 and user display section 72.

The user setting call button 75 is constructed of a plurality of buttons 75a, 75b and 75c as shown in FIG. 10 and the buttons 75a, 75b and 75c correspond to the respective users who are set and registered in advance respectively.

Now FIG. 10 shows a “01: User 01” button 75a corresponding to “user 01,” a “02: User 02” button 75b corresponding to “user 02” and a “03: User 03” button 75c corresponding to “user 03” among the user settings registered in advance, and pressing any one button causes the corresponding user setting to be called and displayed.

Thus, the present second embodiment provides an effect that when calling a user setting which is set and registered in advance, it is possible to call the user setting more easily.

### Third Embodiment

Next, a third embodiment of the present invention will be described.

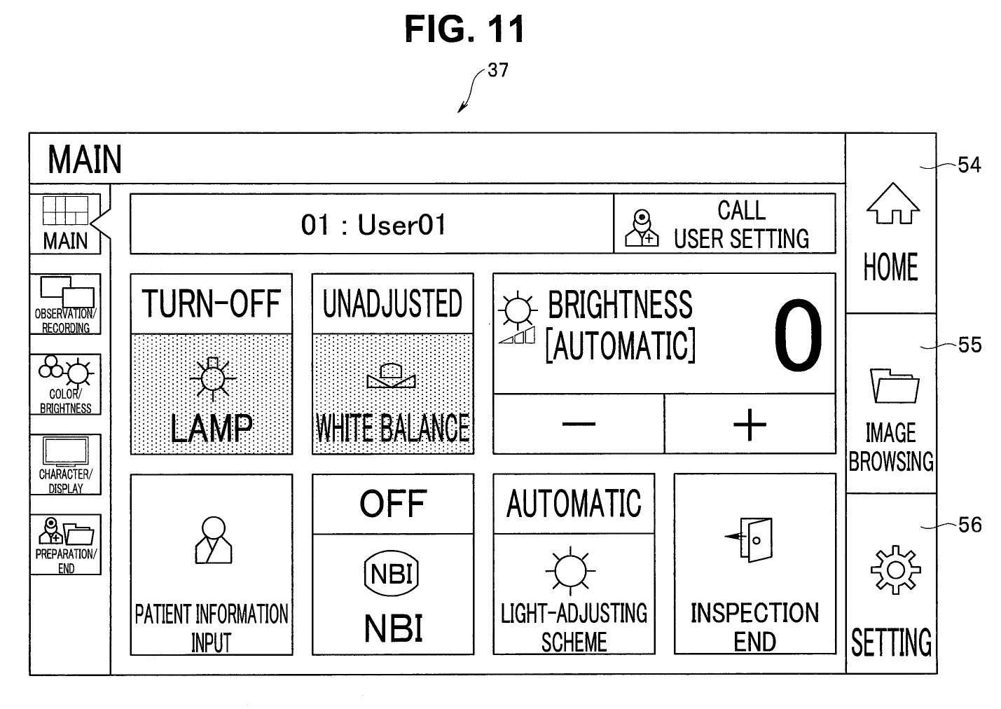

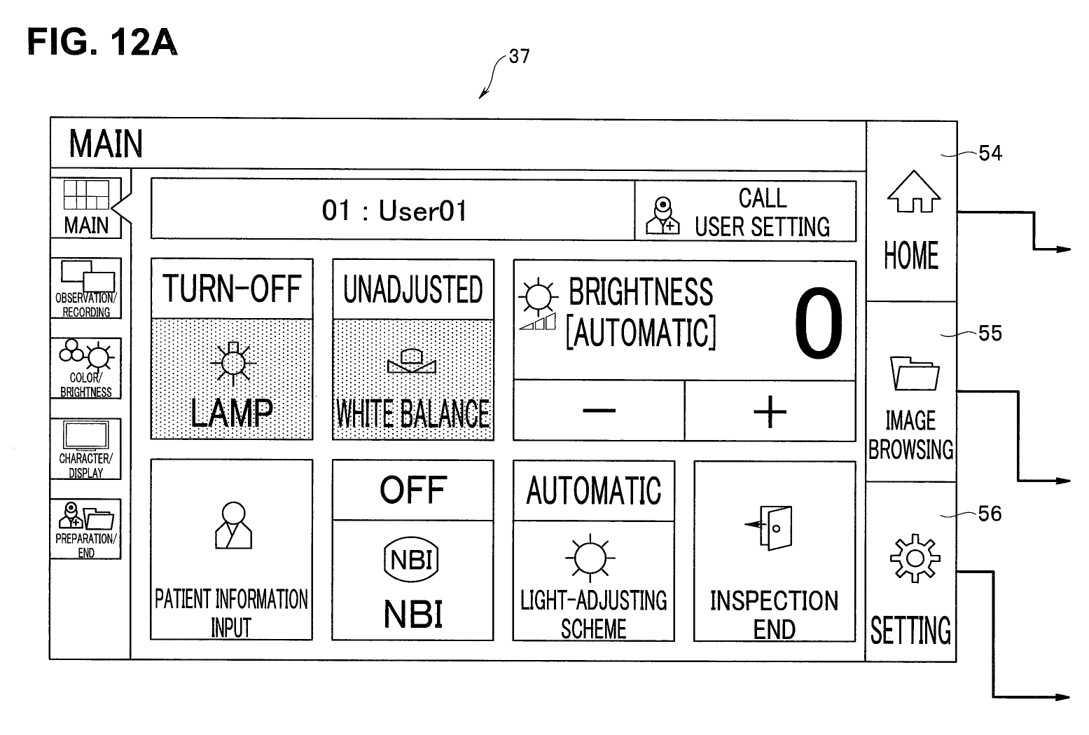

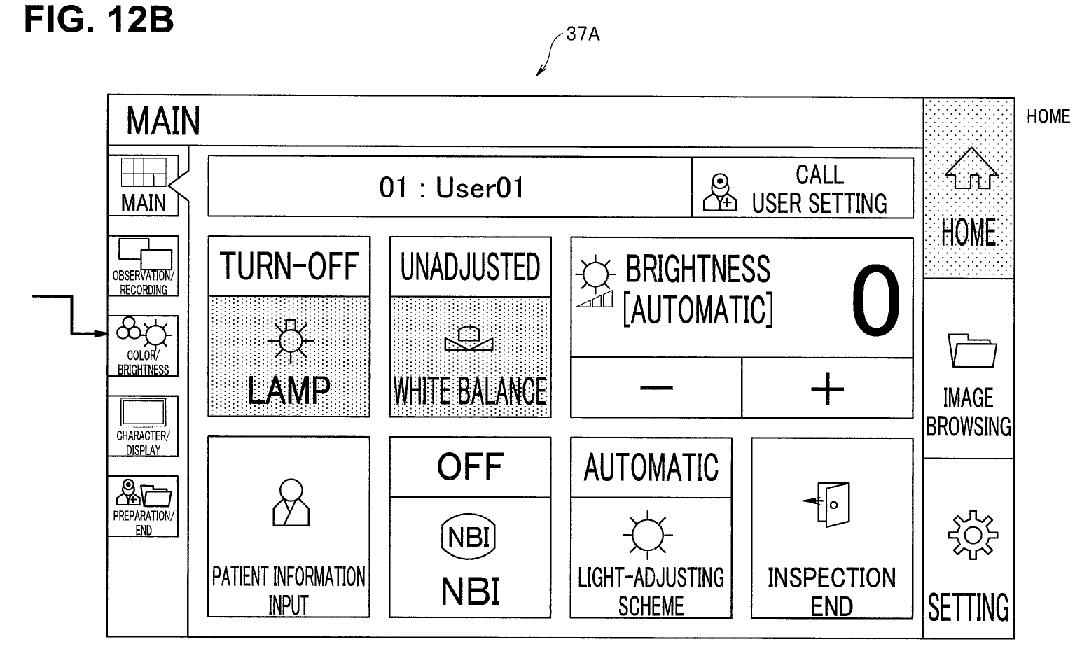

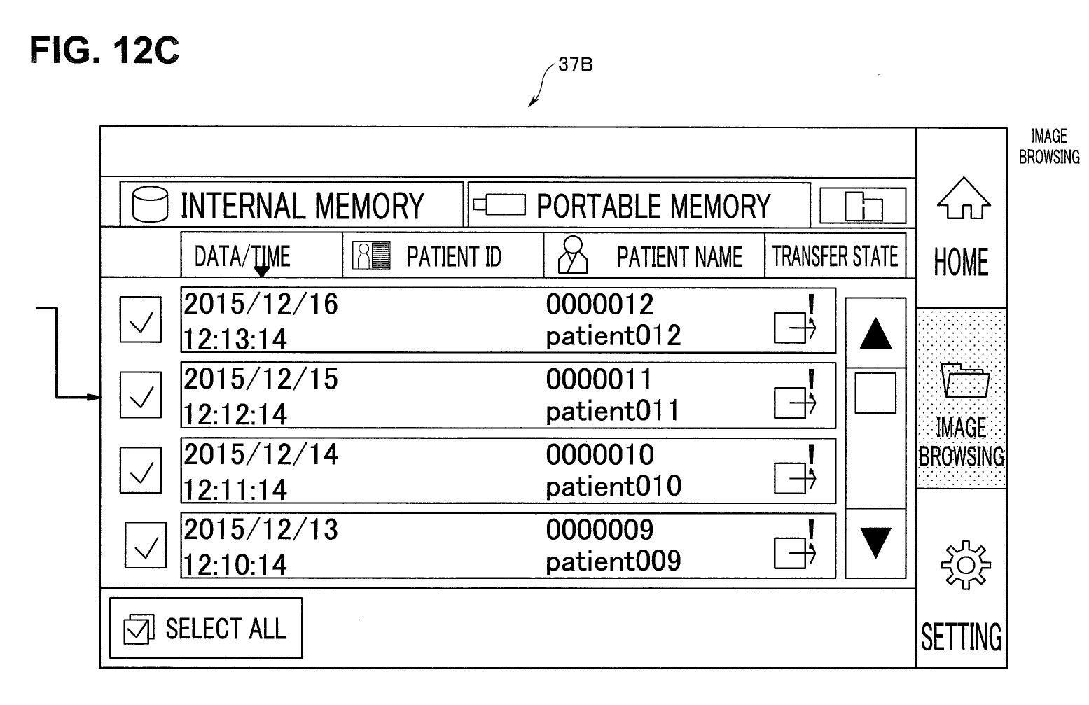

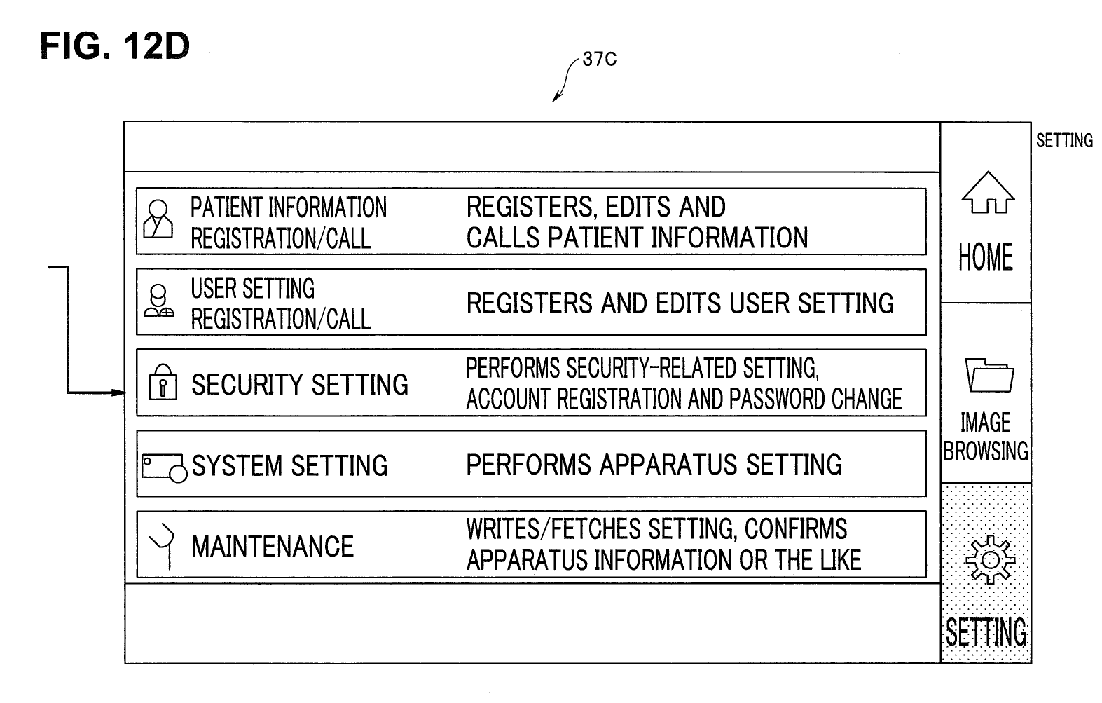

FIG. 11 is a diagram illustrating a display example of a touch panel of a video processor in an endoscope system according to the third embodiment of the present invention, FIG. 12A is diagram illustrating a display example of the touch panel of the video processor in the endoscope system according to the third embodiment, FIG. 12B is a diagram illustrating a display example of the touch panel when a home button is pressed on the touch panel in the endoscope system according to the third embodiment, FIG. 12C is a diagram illustrating a display example of the touch panel when an image browsing button is pressed on the touch panel in the endoscope system according to the third embodiment and FIG. 12D is a diagram illustrating a display example of the touch panel when a setting button is pressed on the touch panel in the endoscope system according to the third embodiment.

The endoscope system according to the present third embodiment has basically the same configuration as the configuration of the first embodiment, and so only differences from the first embodiment will be described here and description of other details will be omitted.

Conventionally, a video processor which is a signal processing apparatus includes many functions and setting items and is configured to be able to select and execute the functions and setting items that suit the purpose. Many of the functions and settings make it necessary to open a menu and go through several layers to reach a desired state. In other words, many operation steps are required and it has been difficult to search a function or setting that suits the purpose.

In view of such circumstances, the endoscope system according to the present third embodiment is configured to classify functions and settings included in the video processor 3 depending on a usage scene, provide a selection button corresponding to the usage scene and easily select a function or setting corresponding to the scene.

As shown in FIG. 11, on the display screen of the touch panel 37 according to the present embodiment, the home button 54, the image browsing button 55 and the setting button 56 are disposed on a side of the function operation portion region 53 in addition to the status display section region 51, the function classification switching display section region 52 and the function operation portion region 53.

As shown in FIG. 12A to FIG. 12D, when pressed (touched), the home button 54 is configured to display a screen 37A to be used during an endoscope inspection.

When pressed (touched), the image browsing button 55 is configured to display a screen 37B to be used for image browsing including data arrangement after the endoscope inspection.

Furthermore, when pressed (touched), the setting button 56 is configured to display a screen 37C for making various settings (e.g., user setting, maintenance or system setting) when setting the video processor 3 or before an inspection.

According to the endoscope system of the present third embodiment, it is possible to facilitate a selection of a function or setting depending on the usage scene.

### Fourth Embodiment

Next, a fourth embodiment of the present invention will be described.

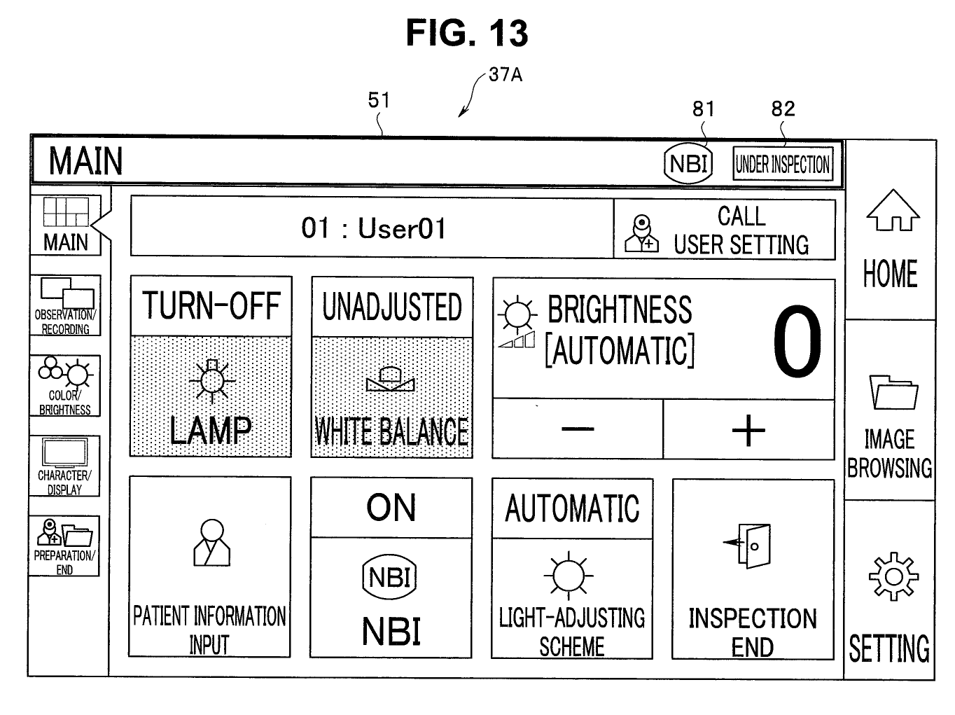

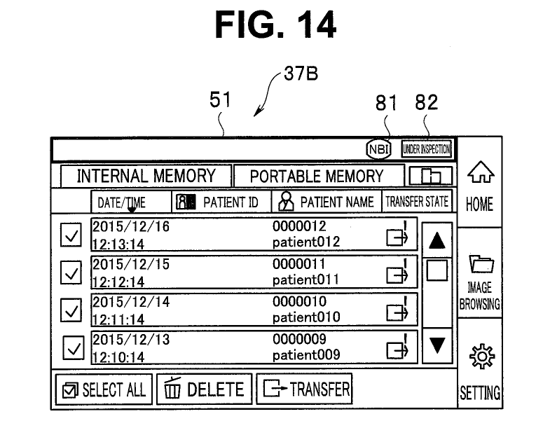

FIG. 13 is a diagram illustrating a display example of a touch panel of a video processor in an endoscope system according to the fourth embodiment of the present invention and FIG. 14 is a diagram illustrating another display example of the touch panel of the video processor in the endoscope system according to the fourth embodiment.

The endoscope system according to the present fourth embodiment has basically the same configuration as the configuration of the first embodiment, and so only differences from the first embodiment will be described and description of other details will be omitted here.

Conventionally, a video processor or light source apparatus is provided with a display function such as an LED for visually indicating the current status on the front panel and is configured to be able to grasp the status of the apparatus using the display functions.

On the other hand, these days, an example where a touch panel is disposed on the front face part of the housing as an apparatus operation display device is known among apparatuses such as video processors.

In the case of the apparatus adopting a touch panel, many functions can be easily displayed because of a high degree of freedom of the GUI, whereas in the prior art, the display function that visually displays the aforementioned current status of the apparatus is not configured to be “always” displayed.

In view of such circumstances, the endoscope system of the present fourth embodiment is configured to provide a region for “always” displaying the current status of the apparatus on the display screen of the touch panel 37 included in the video processor 3 irrespective of other screens that vary successively.

As shown in FIG. 13, in the present fourth embodiment, a region for “always” displaying the current status of the apparatus is disposed in part of the status display section region 51 on the display screen of the touch panel 37.

To be more specific, suppose that the video processor 3 is now set in an NBI observation mode and that an inspection is currently in progress in the NBI observation mode.

At this time, when the home button 54 is pressed and the main screen is displayed as the display screen of the touch panel 37 as shown in FIG. 13 (touch panel 37A), an NBI observation mode mark 81 indicating that the video processor 3 is set in the NBI observation mode and an under-inspection mark 82 indicating that an inspection is currently in progress in the NBI observation mode are displayed on the right side part of the status display section region 51.

On the other hand, when the image browsing button 55 is pressed and the image browsing screen is displayed as the display screen of the touch panel 37 as shown in FIG. 14 (touch panel 37B), while a predetermined screen associated with image browsing is displayed for the region corresponding to the function operation portion region 53, the NBI observation mode mark 81 indicating that the video processor 3 is set in the NBI observation mode and the under-inspection mark 82 indicating that an inspection is currently in progress in the NBI observation mode are displayed on the right side part of the status display section region 51 as in the case where the main screen in FIG. 13 is displayed.

According to the endoscope system of the present fourth embodiment, it is possible to “always” display the current status of the apparatus on the display screen of the touch panel 37 included in the video processor 3 irrespective of other screens that successively vary.

### Fifth Embodiment

Next, a fifth embodiment of the present invention will be described.

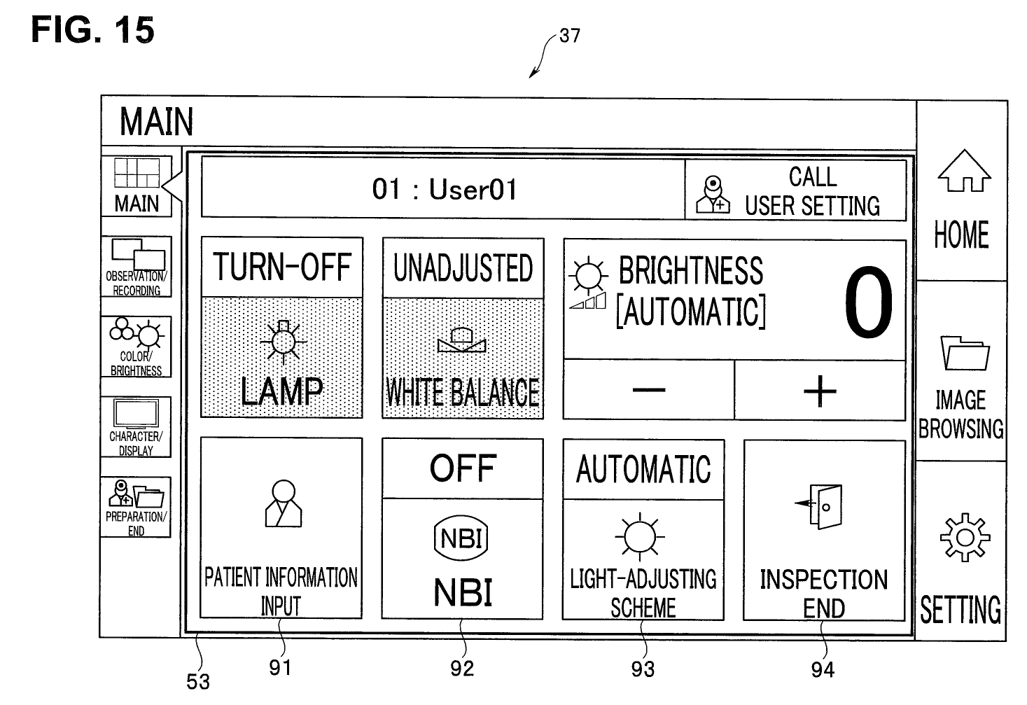

FIG. 15 is a diagram illustrating a display example of a touch panel of a video processor in an endoscope system according to the fifth embodiment of the present invention.

The endoscope system according to the present fifth embodiment has basically the same configuration as the configuration of the first embodiment, and so only differences from the first embodiment will be described here and description of other details will be omitted.

Conventionally, a video processor which is a signal processing apparatus is provided with many functions and an operation keyboard connected to the video processor is provided with function keys corresponding to the functions.

On the other hand, as described above, an example is known these days among apparatuses such as video processors where a touch panel is disposed on the front face part of the housing as an operation display device for the apparatus, and in the case of the apparatus adopting the touch panel, it is possible to easily display many functions due to a high degree of freedom of the GUI, whereas since a menu of a hierarchical structure is used for convenience of the display region, many operation steps are needed to reach a desired function.

In view of such circumstances, the endoscope system of the present fifth embodiment is configured to provide an arbitrary function button on the topmost screen first displayed on the touch panel 37 of the video processor 3 to facilitate a selection of a function depending on a scene.

As shown in FIG. 15, in the present fifth embodiment, the function operation portion region 53 on the display screen of the touch panel 37 is provided with a region in which a custom button to which a function can be freely assigned can be disposed.

That is, as shown in FIG. 15, custom buttons 91 to 94 to which an arbitrary “function” set by a user setting can be assigned are provided at the bottom side part of the function operation portion region 53 on the main screen that can be displayed as the initial screen.

To be more specific, in the present embodiment, a “patient information input button” is assigned as the custom button 91, an “NBI observation mode setting button” is assigned as the custom button 92, a “light-adjusting scheme setting button” is assigned as the custom button 93 and an “inspection end button” is assigned as the custom button 94.

According to the endoscope system of the present fifth embodiment, it is possible to dispose desired function buttons on the display screen of the initial state and thereby reduce operation steps.

### Sixth Embodiment

Next, a sixth embodiment of the present invention will be described.

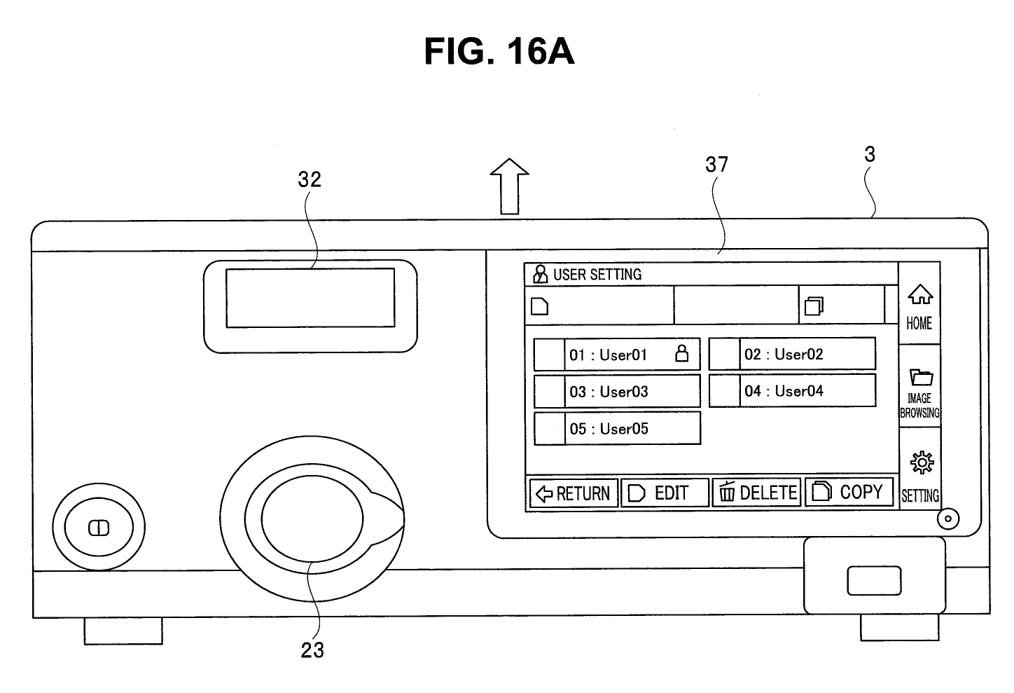

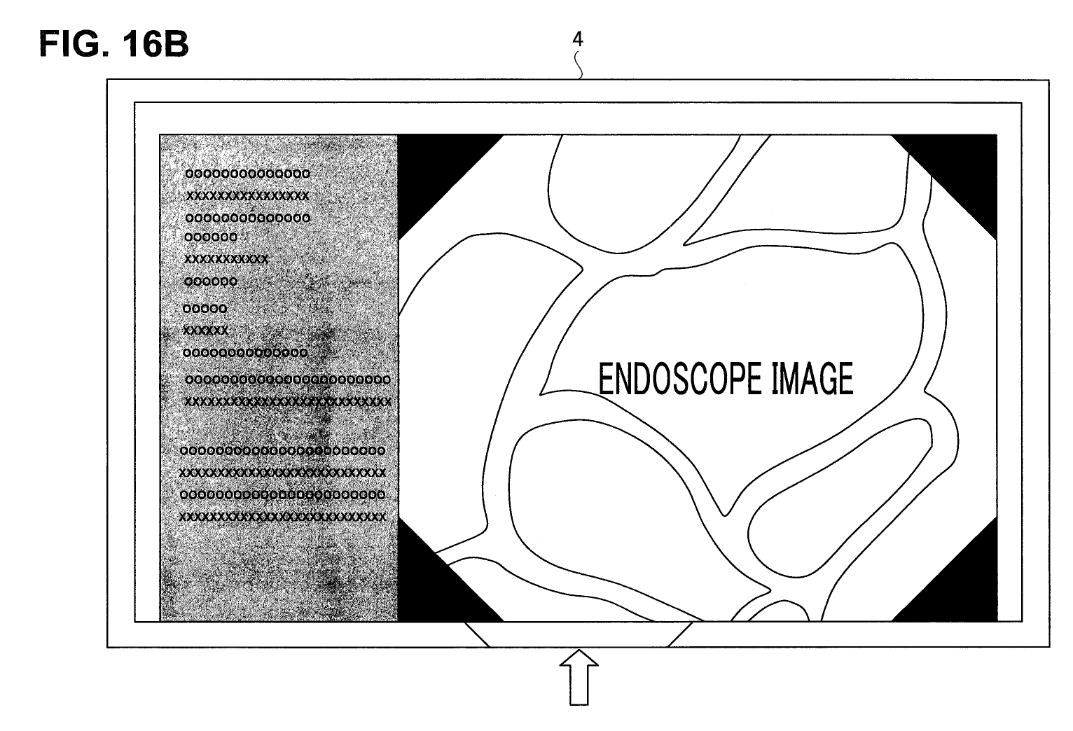

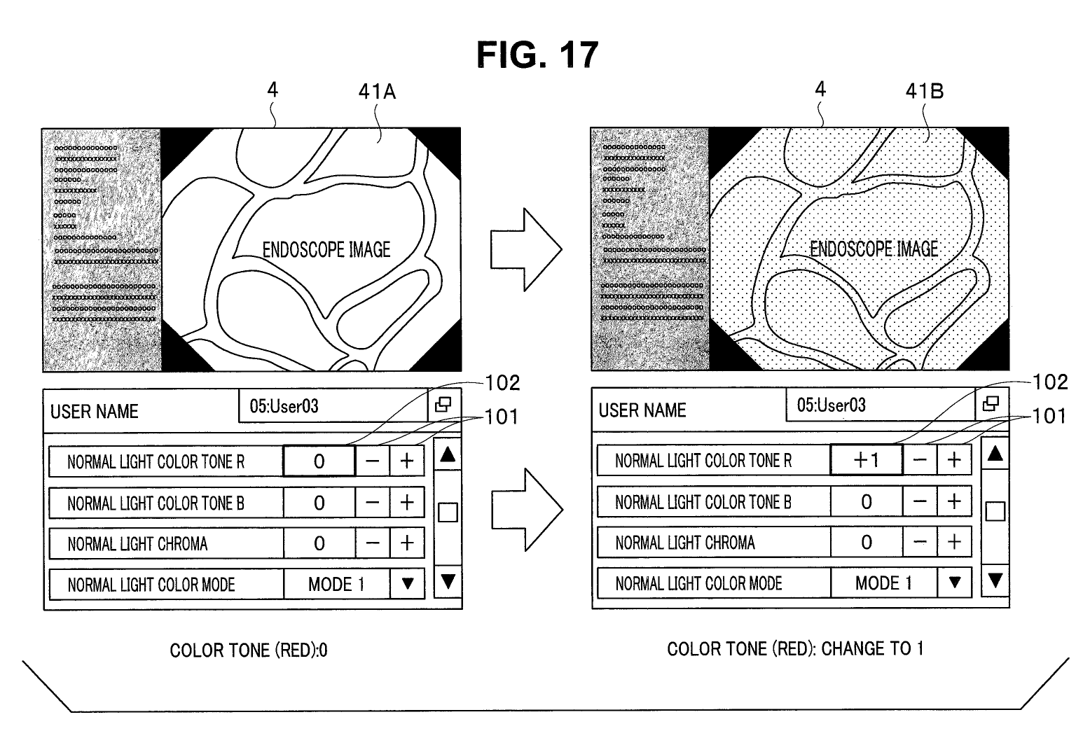

FIG. 16A is a diagram illustrating a video processor in an endoscope system according to a sixth embodiment of the present invention, FIG. 16B is a diagram illustrating an observation monitor in an endoscope system according to a sixth embodiment of the present invention and FIG. 17 is a diagram illustrating a situation in which the display of the observation monitor is changed through a predetermined operation of the touch panel of the video processor in the endoscope system according to the sixth embodiment.

The endoscope system of the present sixth embodiment has basically the same configuration as the configuration of the first embodiment, and so only differences from the first embodiment will be described here and description of other details will be omitted.

Conventionally, when performing an operation associated with a predetermined function on a touch panel of a video processor, if, for example, a set value displayed on the touch panel is changed to change a color tone displayed on the observation monitor 4, the changed set value is not reflected in the apparatus unless the changed set value is saved, and so inconvenience that the set value is dropped from a set value change screen every time a saving operation is performed exists.

In view of such circumstances, the endoscope system of the present sixth embodiment is configured such that, when a predetermined set value change operation is performed on the touch panel 37 of the video processor 3, the set value change result is reflected in various corresponding circuits in real time.

The observation monitor 4 as shown in FIG. 16B is connected to the video processor 3 as shown in FIG. 16A and in the present embodiment, for example, a color tone in an endoscope image displayed on the observation monitor 4 can be changed to an arbitrary set value through an operation of the touch panel 37 of the video processor 3.

To be more specific, suppose that the normal light observation mode is selected now and the color tone of an endoscope image 41A displayed on the observation monitor 4 is in a default state. Here, in the case where a red color tone of the endoscope image 41A of the parameter set stored as the user setting is changed, the user presses the setting button 56 on the touch panel 37 to call the user account to be changed and edits the color tone setting.

In the editing operation, a color tone setting screen as shown on a bottom row in FIG. 17 is displayed on the display screen of the touch panel 37. Note that since the color tone of the endoscope image 41A is in a default state, the set value of the red color tone is “0” on the color tone setting screen and “0” is displayed on a set value display section 102 (see the display screen of the touch panel 37 on the left side on the bottom row in FIG. 17).

After that, the user operates a plus/minus button 101 toward the plus side to increase the set value of the red color tone, “+1” is displayed on the set value display section 102 as shown on the display screen of the touch panel 37 on the right side on the bottom row in FIG. 17 and the set value is incremented.

In the present embodiment, the control section 31 controls the video processing section 33 of the video processor 3 according to the operation of the plus/minus button 101 and applies a process of changing the color tone of the inputted video signal.

Accordingly, the color tone of the endoscope image displayed on the observation monitor 4 is also changed in real time, that is, a new endoscope image 41B with the changed red color tone is displayed in the current case.

When the endoscope image 41B is changed to a user-desired color tone through the operation, the changed parameter is saved and the editing is ended.

According to the endoscope system of the present sixth embodiment, when a predetermined set value change operation is performed on the touch panel 37 of the video processor 3, the set value change result is reflected in real time in various corresponding circuits, making the set value changing/editing operation easier.

According to the present invention, it is possible to provide an endoscope system capable of easily calling a user setting which is registered in advance.

The present invention is not limited to the aforementioned embodiments but can be changed or modified, and so on in various ways without departing from the spirit and scope of the present invention.

What is claimed is:

1. An endoscope processor comprising:
a storage section configured to store a parameter set which is a combination of parameters associated with various processes during an endoscope inspection and set for each user, the storage section being able to store a number of parameter sets corresponding to a plurality of user accounts;
a display section configured to selectively display a display screen made up of a plurality of layers; and
a display control section configured to cause the display section to display a user setting selection list for calling the parameter set for each user stored in the storage section for part of a display region of a main screen that classifies and displays functions with high use frequency during the endoscope inspection.

2. The endoscope processor according to claim 1, wherein the display section comprises a touch pad section including a position information input function.
